(19) United States
(12) Patent Application Publication
(10) Pub. No.: US 2024/0366383 A1
(43) Pub. Date: Nov. 7, 2024

(54) IMPLANTED PIEZOELECTRIC BONE MATERIAL

(71) Applicant: NATIONAL TAIWAN UNIVERSITY, Taipei City (TW)

(72) Inventors: JAW-LIN WANG, Taipei City (TW); YA-CHERNG CHU, New Taipei City (TW)

(21) Appl. No.: 18/355,390

(22) Filed: Jul. 19, 2023

(30) Foreign Application Priority Data
May 5, 2023 (TW) 112116678

(51) Int. Cl. A61F 2/28

(52) U.S. Cl. CPC A61F 2/28; A61F 2002/2864; A61F 2002/30235; A61F 2310/00185

(57) ABSTRACT

An implanted piezoelectric bone material includes a main body and a piezoelectric material. The main body is a hollow pillar or a solid pillar. When the main body is the hollow pillar, the hollow pillar has an inner side wall at a center of the hollow pillar and an outer side wall at an outer side of the hollow pillar. When the main body is the solid pillar, the solid pillar only has the outer side wall at an outer side of the solid pillar. The piezoelectric material is in contact with at least one of the inner side wall and the outer side wall.

### CROSS-REFERENCE TO RELATED PATENT APPLICATION

This application claims the benefit of priority to Taiwan Patent Application No. 112116678, filed on May 5, 2023. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to an implanted bone material, and more particularly to an implanted piezoelectric bone material.

### BACKGROUND OF THE DISCLOSURE

When the bone is damaged due to aging or impact from accidents, surgeons often use implanted bone materials to aid bone healing. A metallic bone material has excellent properties, such as high strength, easy processing and formation, and high reliability, and is thus widely used in orthopedic surgeries. Specifically, the metallic bone material is used in large quantity in biomedical materials for bones and teeth (which need to withstand a high load).

However, in the conventional technology, the state of healing after use of the metallic bone material is not uniform. This is because the existing metallic bone material only aids in bone fixation, but does not help the bone heal in an effective manner. With the advancement of medical technology, the requirements for repair of bone injuries have gradually increased.

Therefore, how to enhance the bone healing effect of the implanted bone materials through an improvement in structural design, so as to overcome the above-mentioned problems, has become one of the important issues to be addressed in the industry.

### SUMMARY OF THE DISCLOSURE

In response to the above-referenced technical inadequacies, the present disclosure provides an implanted piezoelectric bone material. In relevant researches, effects of piezoelectric stimulation on chondrocytes and bone marrow stem cells have been observed. Specifically, mesenchymal stem cells and chondrocytes play a major role before or during an endochondral phase of fracture healing. In order to study the effects of piezoelectric stimulation on bone marrow mesenchymal stem cells (BMMSCs), ultrasound having an intensity range of from 1 mW/cm² to 20 mW/cm² is delivered to mesenchymal stem cells (MSCs) implanted on a quartz cover glass. When the quartz cover glass vibrates under stimulation, an additional local electrical charge is applied to the cell. The results show that the piezoelectric stimulation drives aggregation of MSCs, thereby promoting cartilage formation of MSCs without use of a differentiation culture medium. It should be noted that although both ultrasound and piezoelectric stimulation can upregulate SOX9 protein levels, only the piezoelectric stimulation promotes aggregation and chondrogenic differentiation of BMMSCs.

In order to solve the above-mentioned problems, one of the technical aspects adopted by the present disclosure is to provide an implanted piezoelectric bone material. The implanted piezoelectric bone material includes a main body and a piezoelectric material. The main body is a hollow pillar or a solid pillar. When the main body is the hollow pillar, the hollow pillar has an inner side wall at a center of the hollow pillar and an outer side wall at an outer side of the hollow pillar. When the main body is the solid pillar, the solid pillar only has the outer side wall at an outer side of the solid pillar. The piezoelectric material is in contact with at least one of the inner side wall and the outer side wall.

In one of the possible or preferred embodiments, the main body is made of a metallic material.

In one of the possible or preferred embodiments, the piezoelectric material is ceramics.

In one of the possible or preferred embodiments, the piezoelectric material covers the outer side wall.

In one of the possible or preferred embodiments, the piezoelectric material covers the outer side wall and the inner side wall.

In one of the possible or preferred embodiments, the piezoelectric material is filled in the center of the hollow pillar and is in contact with the inner side wall.

In one of the possible or preferred embodiments, the piezoelectric material is filled in the center of the hollow pillar and covers the outer side wall.

In one of the possible or preferred embodiments, based on a total content of the implanted piezoelectric bone material being 100 vol %, a content of the piezoelectric material is at least 20 vol %.

In one of the possible or preferred embodiments, the main body is made of pure titanium, titanium alloy, or stainless steel.

In one of the possible or preferred embodiments, the piezoelectric material is selected from the group consisting of BaTiO₃, PbTiO₃, Pb(ZrTi)O₃, Na_{0.5}K_{0.5}NbO₃, BaₓSr₁₋ₓNb₂O₅, and Bi_{0.5}Na_{0.5}TiO₃.

In one of the possible or preferred embodiments, the piezoelectric material is piezoelectric plastic.

Therefore, in the implanted piezoelectric bone material provided by the present disclosure, by virtue of “the implanted piezoelectric bone material including a main body and a piezoelectric material, and the main body being a hollow pillar or a solid pillar,” “when the main body is the hollow pillar, the hollow pillar having an inner side wall at a center of the hollow pillar and an outer side wall at an outer side of the hollow pillar,” “when the main body is the solid pillar, the solid pillar only having the outer side wall at an outer side of the solid pillar,” and “the piezoelectric material being in contact with at least one of the inner side wall and the outer side wall,” an implanted bone material can have an improved effect on bone repairing.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a schematic view of an implanted piezoelectric bone material according to a first embodiment of the present disclosure;

FIG. 2 is a schematic view of the implanted piezoelectric bone material according to a second embodiment of the present disclosure;

FIG. 3 is a schematic view of the implanted piezoelectric bone material according to a third embodiment of the present disclosure;

FIG. 4 is a schematic view of the implanted piezoelectric bone material according to a fourth embodiment of the present disclosure;

FIG. 5 is a schematic view of the implanted piezoelectric bone material according to a fifth embodiment of the present disclosure; and

FIG. 6 is a bar chart showing a comparison of mouse tibial bone strengths in different groups.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first”, “second” or “third” can be used to describe various components, signals or the like, which are for distinguishing one component/signal from another one only, and are not intended to, nor should be construed to impose any substantive limitations on the components, signals or the like.

Based on the concept that piezoelectric stimulation can effectively aid bone healing, various embodiments of a piezoelectric bone material are provided in the present disclosure. After the piezoelectric bone material is implanted into the human body, the piezoelectric stimulation generated from load bearings on the piezoelectric bone material can facilitate bone healing. The implanted piezoelectric bone material in the embodiments of the present disclosure will be described in detail as follows.

### First Embodiment

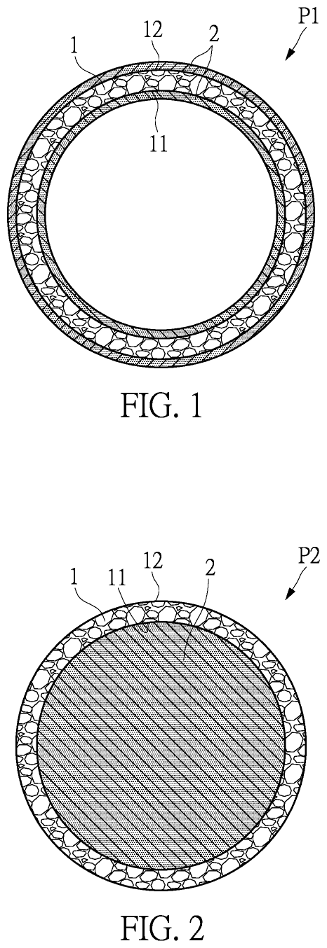

Referring to FIG. 1, a first embodiment of the present disclosure provides a cross-sectional view of an implanted piezoelectric bone material P1. The implanted piezoelectric bone material P1 includes a main body 1 and a piezoelectric material 2. The main body 1 is a hollow pillar, and the hollow pillar has an inner side wall 11 at a center of the hollow pillar and an outer side wall 12 at an outer side of the hollow pillar. The main body 1 can be made of a metallic material. In one embodiment of the present disclosure, the material of the main body 1 can be pure titanium, titanium alloy, or stainless steel. However, the examples above describe only one of the embodiments of the present disclosure, and the present disclosure is not intended to be limited thereto.

In the first embodiment of the present disclosure, the piezoelectric material 2 is coated on the inner side wall 11 and the outer side wall 12 of the main body 1, and then the piezoelectric material 2 undergoes a polarization treatment, so that the implanted piezoelectric bone material P1 is obtained. The piezoelectric material 2 is capable of converting mechanical energy into electrical energy. When a compression force or an extension force is exerted on the piezoelectric material 2, a direct piezoelectric effect is produced, so that the piezoelectric material 2 generates the electrical energy. In practical applications, the piezoelectric bone material of the present disclosure is implanted into the bone. When forces are exerted on the piezoelectric bone material, the piezoelectric effect stimulates osteocytes to proliferate, so as to facilitate bone healing.

Furthermore, the piezoelectric material 2 can be ceramic. For example, the piezoelectric material 2 can be a barium titanate piezoelectric ceramic, a multinary piezoelectric ceramic, or a metaniobate piezoelectric ceramic. However, the examples above describe only one of the embodiments of the present disclosure, and the present disclosure is not intended to be limited thereto. Specifically, the piezoelectric material 2 can be selected from the group consisting of BaTiO₃, PbTiO₃, Pb(ZrTi)O₃ (PZT), Na_{0.5}K_{0.5}NbO₃ (LNK), BaₓSr₁₋ₓNb₂O₅, and Bi_{0.5}Na_{0.5}TiO₃ (BNT). In one exemplary embodiment, the piezoelectric material 2 can be PZT. In addition, the piezoelectric material 2 of the present disclosure can be piezoelectric plastic, such as polyvinylidene difluoride (PVDF).

### Second Embodiment

Referring to FIG. 2, a second embodiment of the present disclosure provides a cross-sectional view of an implanted piezoelectric bone material P2 that is substantially the same as the aforementioned first embodiment, and differences therebetween are described as follows.

In this embodiment, the piezoelectric material 2 is filled in a center of the main body 1. That is, the piezoelectric material 2 is filled in a center of the hollow pillar and undergoes the polarization treatment, so that the implanted piezoelectric bone material P2 is obtained. Therefore, the main body 1 is substantially a solid pillar, and the mechanical strength of the implanted piezoelectric bone material P2 can be further increased.

### Third Embodiment

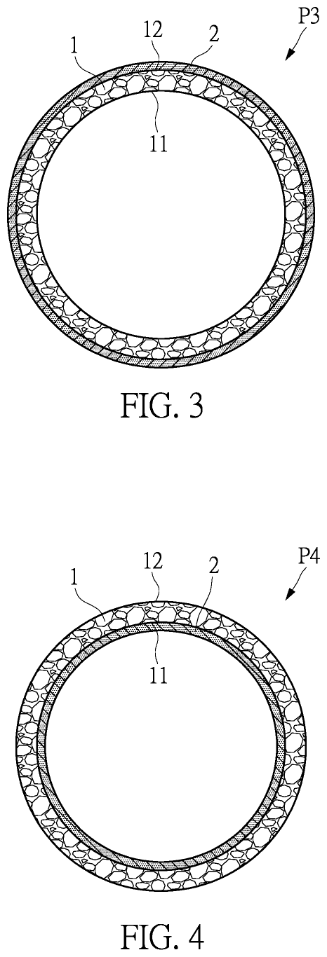

Referring to FIG. 3, a third embodiment of the present disclosure provides a cross-sectional view of an implanted piezoelectric bone material P3 that is substantially the same as the aforementioned first embodiment, and differences therebetween are described as follows.

In this embodiment, the piezoelectric material 2 can be coated only on the outer side wall 12 of the main body 1, and the piezoelectric material 2 undergoes the polarization treatment, so that the implanted piezoelectric bone material P3 is obtained. Therefore, the piezoelectric effect can be generated by a contraction of the muscles with forces exerted on the bone, or by stimulation from an external ultrasonic wave. Then, the generated piezoelectricity can rapidly contact surrounding osteocytes to stimulate osteocyte proliferation. In another embodiment of the present disclosure, the main body 1 can be a solid pillar, and the piezoelectric material 2 can be coated on the outer side wall 12 of the solid pillar.

### Fourth Embodiment

Referring to FIG. 4, a fourth embodiment of the present disclosure provides a cross-sectional view of an implanted piezoelectric bone material P4 that is substantially the same as the aforementioned first embodiment, and differences therebetween are described as follows.

In this embodiment, the piezoelectric material 2 can be coated only on the inner side wall 11 of the main body 1, and the piezoelectric material 2 undergoes the polarization treatment, so that the implanted piezoelectric bone material P4 is obtained. Since the implanted piezoelectric bone material P4 has a light weight, the piezoelectric stimulation generated thereby is suitable for treating bones with a minor injury.

### Fifth Embodiment

Referring to FIG. 5, a fifth embodiment of the present disclosure provides a cross-sectional view of an implanted piezoelectric bone material P5 that is substantially the same as the aforementioned first embodiment, and differences therebetween are described as follows.

In this embodiment, the piezoelectric material 2 is filled in the center of the main body 1, and is coated on the outer side wall 12 of the main body 1. Then, the piezoelectric material 2 undergoes the polarization treatment, so that the implanted piezoelectric bone material P5 is obtained. Therefore, the main body 1 is completely covered by the piezoelectric material 2, so as to obtain optimal mechanical strength and optimal piezoelectric effect. In another embodiment of the present disclosure, the piezoelectric material 2 can be simultaneously coated on the inner side wall 11 and the outer side wall 12 of the main body 1, but without fully filling the center of the main body 1.

In one embodiment of the present disclosure, the polarization treatment refers to treating a combination of the main body 1 and the piezoelectric material 2 in a high pressure direct-current oil bath device at a polarization temperature of 100° C. and a polarization voltage of 3,500 V for 20 minutes, so that the main body 1 and the piezoelectric material 2 can be processed into an implanted piezoelectric bone material. In practical applications, the suitable implanted piezoelectric bone material can be selected according to an amount of damage sustained by the bone.

Furthermore, a three-point bending test is performed to evaluate a mechanical performance of mouse tibial bones. In the three-point bending test, a constant force is exerted on a midpoint of a tibia shaft, so as to evaluate a maximum load that can be applied to the tibia before occurrence of bone fracture or bone breakage. In Control Group, a bone that has not sustained damage is used. In Comparative Example, a bone that has sustained damage and has a metallic bone material (which does not include a piezoelectric material) implanted therein is used. In Experimental Example, a bone that has sustained damage and has the implanted piezoelectric bone material P1 of the first embodiment of the present disclosure implanted therein is used.

Male C57BL/6 mice of 8 weeks old are utilized in the experiment. The animal room is maintained at a temperature of from 21° C. to 24° C. and a relative humidity of from 30% to 70%, and the lighting in the animal room is automatically switched on and off at twelve-hour intervals. Sufficient drinking water and standard feed are supplied to the mice for the duration of the experiment.

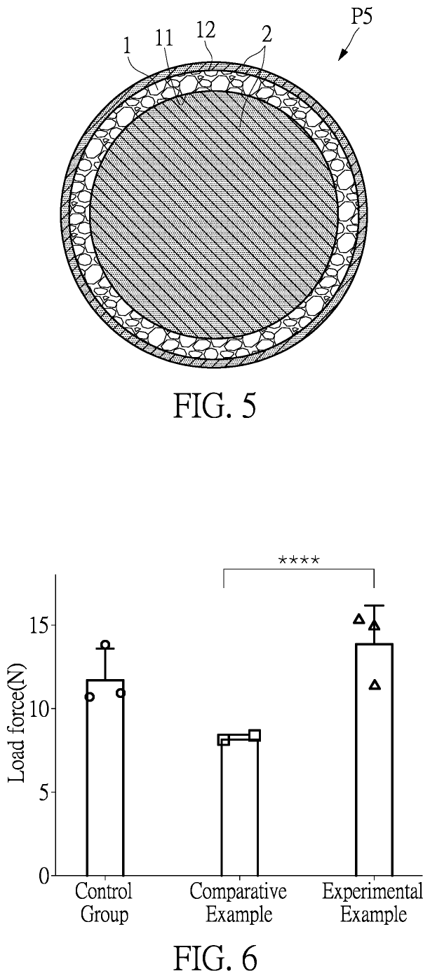

After the mouse is sacrificed, its left tibia is taken, and soft tissues surrounding the left tibia are removed, so that the left tibia can be used on a testing platform to perform the three-point bending test. When the left tibia is fractured, a maximal load (unit: newton) is obtained as the strength of the tibial bone. Reference can be made to FIG. 6, which is a bar chart showing a comparison of tibial bone strengths in the three groups.

Results of a mouse bone fracture experiment measured by the three-point bending test are shown in FIG. 6. The group using the implanted piezoelectric bone material of the present disclosure (Experimental Example) has a higher bone strength as compared to the group using the metallic bone material (Comparative Example), and even has a bone strength higher than that of the group where the bone is uninjured (Control Group). In other words, the implanted piezoelectric bone material of the present disclosure indeed has the effect of stimulating the growth of osteocytes, and can achieve the effect of enhancing bone repair.

### Beneficial Effects of the Embodiments

One of the advantageous effects of the present disclosure is that, in the implanted piezoelectric bone material provided by the present disclosure, by virtue of “the implanted piezoelectric bone material including a main body and a piezoelectric material, and the main body being a hollow pillar or a solid pillar,” “when the main body is the hollow pillar, the hollow pillar having an inner side wall at a center of the hollow pillar and an outer side wall at an outer side of the hollow pillar,” “when the main body is the solid pillar, the solid pillar only having the outer side wall at an outer side of the solid pillar,” and “the piezoelectric material being in contact with at least one of the inner side wall and the outer side wall,” an implanted bone material can have an improved effect on bone repairing.

It is worth mentioning that, although the implanted piezoelectric bone material can also be manufactured in the form of dispersing the piezoelectric material in a bone material, the implanted piezoelectric bone material manufactured in such a manner has poor pressure sensitivity. As a result, a signal strength generated by the piezoelectric material is affected, and other materials such as graphene are required to be added for increasing dielectric and electrical conductive properties, thereby increasing processing complexity and manufacturing costs. The implanted piezoelectric bone material provided by the present disclosure has a simple manufacturing process. By having the piezoelectric material filled in the main body and/or coated on the surface of the main body, the implanted piezoelectric bone material can be manufactured and applied to orthopedic operations to increase a bone healing speed. Therefore, the implanted piezoelectric bone material of the present disclosure has the advantages of having a simple manufacturing process and effectively stimulating the bone to heal.

Furthermore, in the present disclosure, the piezoelectric material can be filled into existing clinical bone materials, and undergo high temperature polarization to introduce piezoelectric characteristics into the existing bone materials. The manufacturing process is not only simple and convenient but is also economically efficient. In addition, implanting the bone material that has the piezoelectric characteristics into patients with bone fracture can provide effective piezoelectric stimulations after operation, so as to facilitate bone healing.

Moreover, in the implanted piezoelectric bone material of the present disclosure, based on a total content of the implanted piezoelectric bone material being 100 vol %, a content of the piezoelectric material is at least 20 vol %. Accordingly, sufficient piezoelectric stimulation can be generated after forces are exerted on the bone material, so as to facilitate bone healing. If the content of the piezoelectric material is less than 20 vol %, it is not possible to provide sufficient piezoelectric stimulation.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. An implanted piezoelectric bone material, comprising:
a main body being a hollow pillar or a solid pillar, wherein, when the main body is the hollow pillar, the hollow pillar has an inner side wall at a center of the hollow pillar and an outer side wall at an outer side of the hollow pillar; wherein, when the main body is the solid pillar, the solid pillar only has the outer side wall at an outer side of the solid pillar; and
a piezoelectric material, wherein the piezoelectric material is in contact with at least one of the inner side wall and the outer side wall.

2. The implanted piezoelectric bone material according to claim 1, wherein the main body is made of a metallic material.

3. The implanted piezoelectric bone material according to claim 1, wherein the piezoelectric material is ceramic.

4. The implanted piezoelectric bone material according to claim 1, wherein the piezoelectric material covers the outer side wall and the inner side wall.

5. The implanted piezoelectric bone material according to claim 1, wherein the piezoelectric material covers the inner side wall.

6. The implanted piezoelectric bone material according to claim 1, wherein the piezoelectric material covers the outer side wall.

7. The implanted piezoelectric bone material according to claim 1, wherein the piezoelectric material is filled in the center of the hollow pillar, and is in contact with the inner side wall.

8. The implanted piezoelectric bone material according to claim 1, wherein the piezoelectric material is filled in the center of the hollow pillar, and covers the outer side wall.

9. The implanted piezoelectric bone material according to claim 1, wherein, based on a total content of the implanted piezoelectric bone material being 100 vol %, a content of the piezoelectric material is at least 20 vol %.

10. The implanted piezoelectric bone material according to claim 1, wherein the main body is made of pure titanium, titanium alloy, or stainless steel.

11. The implanted piezoelectric bone material according to claim 1, wherein the piezoelectric material is selected from the group consisting of BaTiO₃, PbTiO₃, Pb(ZrTi)O₃, Na_{0.5}K_{0.5}NbO₃, BaₓSr₁₋ₓNb₂O₅, and Bi_{0.5}Na_{0.5}TiO₃.

12. The implanted piezoelectric bone material according to claim 1, wherein the piezoelectric material is piezoelectric plastic.
